(19) United States
(12) Patent Application Publication
(10) Pub. No.: US 2009/0105537 A1
(43) Pub. Date: Apr. 23, 2009

(54) Device, System and Method for In-Vivo Examination

(76) Inventors: Daniel Gat, Haifa (IL); Raphael Rabinovitz, Raanana (IL); Sharon Bonfis, Yokneam Ilit (IL)

Correspondence Address: Pearl Cohen Zedek Latzer, LLP, 1500 Broadway, 12th Floor, New York, NY, 10036, US

(21) Appl. No.: 11/794,538

(22) PCT Filed: Jan. 1, 2006
(86) PCT No.: PCT/IL06/00007

§ 371 Date: Feb. 29, 2008

Related U.S. Application Data
(60) Provisional application No. 60/640,096, filed on Dec. 30, 2004.

(51) Int. Cl. A61B 1/04; A61K 33/42; A61P 1/10; A61K 31/765

(52) U.S. Cl. 600/109; 424/606; 424/78.31

(57) ABSTRACT

A device, a system and a method for in-vivo examination. A method for in-vivo examination includes substantially emptying a subject's colon from content, and inserting an autonomous in-vivo imaging device into the subject's gastrointestinal tract. An in-vivo examination kit includes an autonomous in-vivo imaging device, a wireless receiver, an antenna set, a laxative, and optionally a stimulant and an instructions leaflet.

### FIELD OF THE INVENTION

The present invention relates to the field of medical procedures. More specifically, the present invention relates to methods for preparing a subject for an examination of the gastrointestinal tract.

### BACKGROUND OF THE INVENTION

An endoscopic examination of the large intestine (colonoscopy) may be used to look for early signs of cancer in the colon. It may further be used to diagnose the causes of unexplained changes in bowel habits. Colonoscopy may allow a physician to see pathology in the colon, such as inflamed tissue, abnormal growths, polyps, cancers ulcers, and bleeding.

Colonoscopy involves inserting a long, flexible, lighted tube through a patient's rectum and guiding it into the colon. An image of the inside of the colon is obtained, so the physician can examine the lining of the colon. Usually, air is blown into the colon, which inflates the colon and helps the physician see.

Bleeding and puncture of the colon are possible complications of colonoscopy, and it is usually necessary to give the patient pain medication and sedatives to keep him comfortable and relaxed during the colonoscopy. Additionally, the patient's colon must be emptied for the procedure. Typically, a patient follows a liquid diet for one to three days before the examination, and takes a laxative the night before the procedure. This is an unpleasant preparation procedure, which may discourage patients from getting examined.

Another known procedure for examining the gastrointestinal (GI) tract, particularly the colon, is Virtual Colonoscopy (VC). This method uses X-rays and computers to produce two-dimensional and three-dimensional images of the colon and display them on a screen. VC may be performed using Computed Tomography (CT) or using Magnetic Resonance Imaging (MRI). Typically, preparations for VC include the intake of laxatives or other oral agents the day before the procedure, to clear stool from the colon. VC may not involve the hazards and discomfort of other known endoscopic procedures; however, the computer-generated images of a VC procedure may lack details provided by an endoscopic imager.

### SUMMARY OF THE INVENTION

Some embodiments of the invention provide a method for clearing, cleaning, cleansing and/or substantially emptying a person's GI tract. According to some embodiments, a method is provided for substantially emptying a colon.

Some embodiments of the invention provide procedures for preparing a patient for an in-vivo examination of the GI tract. According to some embodiments, a procedure is provided for examination of the large intestine (colon). Some embodiments may allow effective timing for colon cleansing and/or effective timing for propelling an in-vivo imaging device to the colon and in the colon.

Procedures according to some embodiments of the invention may be typically shorter than conventional procedures, may be performed in non hospital environments (e.g., in the patient's home), and/or may facilitate patient compliance. For example, procedures according to some embodiments may include a relatively short fasting period, and may allow an examination of the GI tract to take place overnight during the patient's sleep. According to some embodiments, a procedure may be implemented with or prior to an examination of the GI tract by a swallowable, typically autonomous, in-vivo imaging device. According to other embodiments, a procedure may be implemented with or prior to other GI examination methods, such as endoscopy (e.g., colonoscopy) and other suitable imaging methods or other examination methods.

Some embodiments provide a kit which may include, for example, a laxative, a stimulant, a gut lavage, a swallowable in-vivo imaging device or capsule, a wearable set of receiving antennas, and a receiver/recorder unit for receiving and recording images transmitted by the in-vivo imaging device.

In some embodiments, for example, a method for in-vivo examination may include: substantially emptying a subject's colon from content; and inserting an autonomous in-vivo imaging device into the subject's gastrointestinal tract.

In some embodiments, for example, substantially emptying the subject's colon may include: administering a laxative; waiting a pre-defined time period; and administering a stimulant and/or prokinetic agent.

In some embodiments, for example, the laxative may be a Polyethylene Glycol based laxative.

In some embodiments, for example, the laxative may be a substance which includes polyethylene glycol (PEG) and/or sodium phosphate. According to some embodiments laxatives may be selected from a group consisting of: Soffodex™, Fleet Phospho-soda®, Meroken, GoLYTELY®, NuLYTELY®, Polyethylene Glycol, and Polyethylene Glycol 3350.

In some embodiments, for example, the stimulant may be a substance selected from a group consisting of: Bisacodyl, Senna, and Castor Oil.

In some embodiments, for example, the Prokinetic may be a substance selected from a group consisting of: Tegaserod, Domperidone, and Metoclopramide

In some embodiments, for example, administering the laxative may include: administering a first portion of the laxative; waiting a pre-defined time period; and administering a second portion of the laxative.

In some embodiments, for example, inserting the autonomous in-vivo imaging device may include inserting the autonomous in-vivo imaging device at night time and at least thirty minutes after administering the stimulant. In some embodiments, for example, the method may further include administering a meal and a booster laxative at least five hours after inserting the autonomous in-vivo imaging device.

In some embodiments, for example, inserting the autonomous in-vivo imaging device may include inserting the autonomous in-vivo imaging device at morning time and at least four hours after administering the stimulant. In some embodiments, for example, the method may further include administering a meal and a additional dose of laxative at least one hour after inserting the autonomous in-vivo imaging device.

In some embodiments, for example, the method may further include administering additional dose of laxative only after the autonomous in-vivo imaging device exits the stomach.

In some embodiments, for example, inserting the autonomous in-vivo imaging device may include swallowing a swallowable in-vivo imaging device.

According to one embodiment of the invention the in vivo imaging device has a volume to weight ratio that enables it essentially to float in the colon fluid.

According to one embodiment of the invention the in vivo imaging device has a specific gravity (SG) of about 0.7-0.98 in the colon's fluid.

In some embodiments, for example, method may further include receiving in-vivo images from the autonomous in-vivo imaging device, storing the images, processing the images, and/or displaying the images.

In some embodiments, for example, substantially emptying the subject's colon may include: fasting from noon a day before the in-vivo examination; and administering a laxative. In some embodiments, for example, inserting the autonomous in-vivo imaging device may include inserting the autonomous in-vivo imaging device approximately twelve hours from the beginning of the fasting. In some embodiments, for example, administering the laxative may include: administering a first portion of the laxative approximately two hours from the beginning of the fasting; and administering a second portion of the laxative approximately eight hours from the beginning of the fasting. In some embodiments, for example, the method may further include administering a stimulant approximately one hour prior to inserting the autonomous in-vivo imaging device.

In some embodiments, for example, an in-vivo examination kit may include: an autonomous in-vivo imaging device; a wireless receiver; an antenna set; and a laxative. According to some embodiments the receiver may include a monitor, for example, for on line viewing of the GI tract.

In some embodiments, for example, the laxative may be Polyethylene Glycol based laxative. In some embodiments, for example, the laxative may be a substance selected from a group consisting of: Soffodex™, Phospho-soda®, Meroken, GoLYTELY®, NuLYTELY®, Polyethylene Glycol, and Polyethylene Glycol 3350.

In some embodiments, for example, the laxative may include a first portion of the laxative intended for ingestion at a first time, and a second portion of the laxative intended for ingestion at a second time.

In some embodiments, for example, the in-vivo examination kit may include a stimulant, e.g., Bisacodyl, Senna, and/or Castor Oil.

In some embodiments, for example, the laxative and/or the stimulant, or one or more portions thereof, may be color-coded.

In some embodiments, for example, the in-vivo examination kit may include an instruction article having instructions to ingest the laxative, to wait a pre-defined time period longer than four hours, and to ingest the stimulant.

In some embodiments, for example, the in-vivo examination kit may include an instruction leaflet having instructions to insert the autonomous in-vivo imaging device at night time and at least thirty minutes after administering the stimulant; and optionally, an instruction to ingest a meal and a booster laxative at least five hours after inserting the autonomous in-vivo imaging device.

In some embodiments, for example, the in-vivo examination kit may include an instruction leaflet having instructions to insert the autonomous in-vivo imaging device at morning time and at least four hours after administering the stimulant; and optionally, an instruction to ingest a meal and a booster laxative at least one hour after inserting the autonomous in-vivo imaging device.

In some embodiments, for example, the in-vivo examination kit may include a packaging to enclose the autonomous in-vivo imaging device, the wireless receiver, the antenna set, and the laxative.

In some embodiments, for example, the autonomous in-vivo imaging device may include an imager, an illumination source, and a transmitter.

### BRIEF DESCRIPTION OF THE DRAWINGS

The subject matter regarded as the invention is particularly pointed out and distinctly claimed in the detailed description in the specification. The invention, however, may best be understood by reference to the following detailed description when read with the accompanied drawings in which:

FIG. 1 is a flow chart describing a method for examining a colon, according to some embodiments of the invention;

FIGS. 2A1, 2A2 and 2B are schematic timelines describing procedures according to embodiments of the invention;

FIG. 3 is a schematic illustration of a kit for in-vivo examination according to some embodiments of the invention; and

FIG. 4 is a schematic illustration of an in-vivo system in accordance with some embodiments of the invention.

### DETAILED DESCRIPTION OF THE INVENTION

In the following description, various aspects of the invention are set forth. For purposes of explanation and in order to provide an understanding of the invention, specific configurations and details are set forth in order to provide a thorough understanding of the present invention. However, it will also be apparent to one skilled in the art that the invention may be practiced without the specific details presented herein. Furthermore, well-known features may be omitted or simplified in order not to obscure the invention. Some embodiments of the present invention are directed to a typically swallowable in-vivo sensing device, e.g., a typically swallowable in-vivo imaging device. Devices according to embodiments of the present invention may be similar to embodiments described in U.S. patent application Ser. No. 09/800,470, entitled “Device And System For In-vivo Imaging”, filed on 8 Mar. 2001, published on Nov. 1, 2001 as United States Patent Application Publication Number 2001/0035902, and/or in U.S. Pat. No. 5,604,531 to Iddan et al., entitled “In-Vivo Video Camera System”, and/or in U.S. patent application Ser. No. 10/046,541, filed on Jan. 16, 2002, published on Aug. 15, 2002 as United States Patent Application Publication Number 2002/0109774, all of which are hereby incorporated by reference. An external receiving unit and processor, such as in a workstation, such as those described in the above publications may be suitable for use with embodiments of the present invention. Devices and systems as described herein may have other configurations and/or other sets of components. For example, the present invention may be practiced using an endoscope, needle, stent, catheter, etc. Some in-vivo devices may be capsule shaped, or may have other shapes, for example, a peanut shape or tubular, spherical, conical, or other suitable shapes. Devices and systems as described herein may have other configurations and other sets of components.

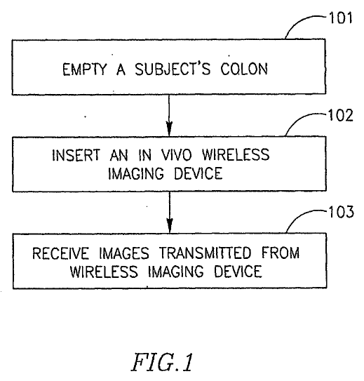

FIG. 1 schematically illustrates a method for examining a body lumen, e.g., the colon, according to some embodiments of the invention. According to some embodiments, a subject's colon may be substantially emptied from content (box 101). The subject may swallow, or may otherwise insert into his GI tract, a wireless, typically autonomous, in-vivo imaging device (box 102) able to image the GI tract and to transmit the images to an external receiving unit. In-vivo images from the GI tract may be received from the in-vivo imaging device (box 103), for example, by an external receiving unit which may be attached onto, or be in proximity with, the subject's body. The image data, for example, may be recorded, displayed, processed, and/or analyzed, e.g., by a professional and/or a computing platform.

According to some embodiments, the autonomous in-vivo imaging device may be floatable, such that it may be carried through body lumens (e.g., the colon) together with movement of liquids in those lumens.

According to some embodiments, a swallowable in-vivo imaging device may include a delay mechanism, such as pH, temperature, conductivity, shear, pressure and so on or may be activated or fully-activated or triggered into action in a certain environment or after a certain amount of time; for example, the in-vivo device may save power or reduce power consumption during the passage through the body lumen, and may be activated or fully-activated only at the area-of-interest intended for examination.

According to some embodiments, the operation of emptying the subject's colon may include, for example, intake of laxatives, prokinetic agents and/or other stimulants. Stimulants and/or laxatives may encourage bowel movements, for example, by acting on the intestinal wall, e.g., by increasing muscle contractions that move along the stool mass.

In another embodiment there is also provided a device (for example Freelax™) for an external massage of the abdomen, so as to enhance motility through the lumen.

Stimulants and/or laxatives that may be used according to some embodiments may include, for example, Bisacodyl, Senna, and/or Castor Oil. Other suitable stimulants, laxatives, drugs, medicines, chemicals, agents and/or substances may be used. Optionally, according to some embodiments, the subject may be required to keep a relatively low residue diet, e.g., for one day or two days prior to the examination, for example, in addition to using laxatives.

Optionally, a subject may be required to intake one or more liquids (for example, water, Diet Sprite®, Diet 7-Up®, apple juice, cranberry juice, grape juice, or the like) at any stage of the procedure, e.g., to facilitate and/or enhance the procedure.

According to some embodiments, substantially emptying a subject's GI tract from content may not include emptying the lumen from typically clear liquids, or may include maintaining typically clear liquids in the lumen. For example, a subject's colon may be substantially cleared from stool and/or other substantial content, but may still include endo-luminal liquids or other liquids, e.g., typically clear liquids.

According to some embodiments, the in-vivo device may include, for example, one or more illumination sources, one or more imagers, and an optional optical system, which may positioned behind an optical window and may be contained and/or enclosed within a single housing or shell. The in-vivo device may further include, for example, enclosed within the same housing, a transmitter for transmitting images to an external receiving unit, and a power source (e.g., one or more batteries). In some embodiments, the in-vivo (e.g., swallowable) imaging device may be pushed through the GI tract by the natural action of peristalsis. According to some embodiments, the in-vivo imaging device may be buoyant, and may be carried through a lumen by liquids in the lumen. In some embodiments, after being swallowed or otherwise inserted, the in-vivo device may pass through the esophagus, stomach, duodenum, several meters of the small bowl, the cecum, and may reach the ascending, traverse and descending parts of the colon. Thus, the in-vivo imaging device may be swallowed or otherwise inserted into the GI tract several hours prior to the actual imaging of the colon or of another remote part of the GI tract. In some embodiments, the operation of emptying the colon may be preformed, for example, prior to or after the insertion of the in-vivo device, e.g., while the in-vivo device moves through the GI tract.

According to some embodiments, the in-vivo imaging device may be inserted through the rectum into the colon. For example, the in-vivo imaging device may be placed in the Ceacum by an endoscope that is inserted through a patient's rectum. In some embodiments, the in-vivo imaging device may be inserted into the rectum and may be made to move through the colon, e.g., in substantially an opposite direction of that described above. Other movements of the in-vivo device within the GI tract may be possible.

According to some embodiments, a wired or non-wireless in-vivo device (e.g., endoscope or colonoscope) may be used to examine the GI tract. In some embodiments, the endoscope may be brought to the colon at a relatively more precise time than a swallowable in-vivo device; therefore, it may be required that at that time the operation of emptying the colon be substantially completed, and/or that the operation of emptying the colon precede the operation of inserting the endoscope. Various suitable time lines may be used or adjusted to accommodate various embodiments of the invention.

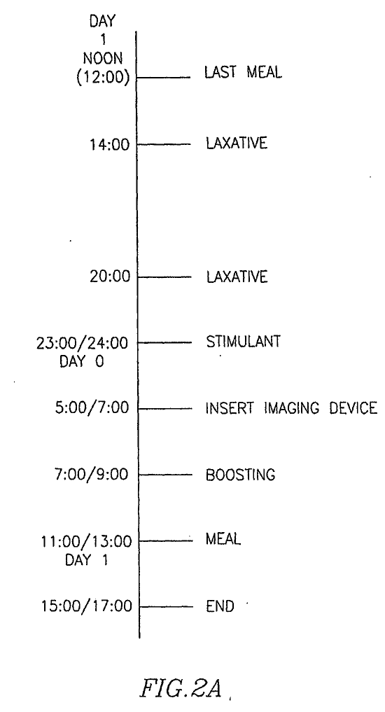

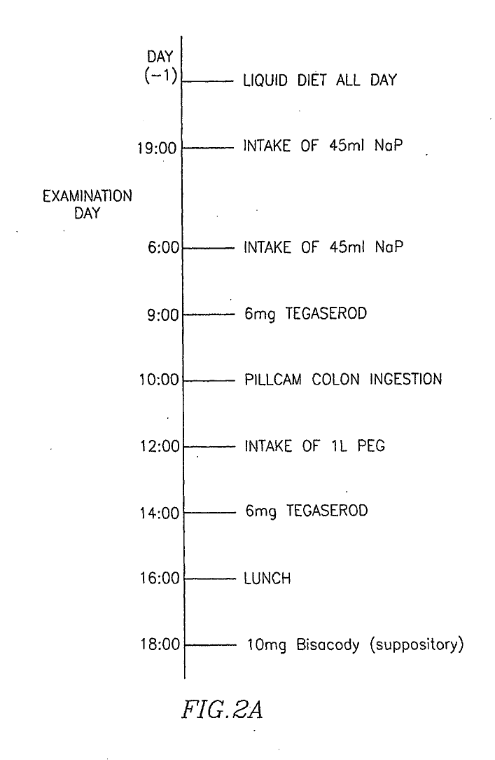

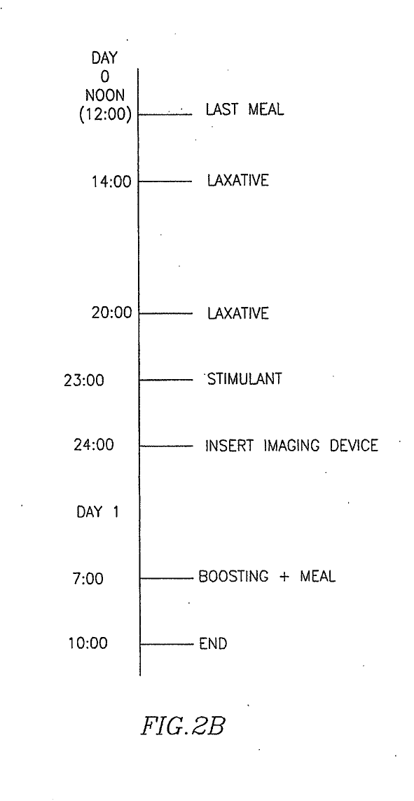

FIGS. 2A1, 2A2 and 2B illustrate a few exemplary time lines for procedures according to some embodiments of the invention; FIGS. 2A1 and 2A2 may correspond to a “day procedure”, whereas FIG. 2B may correspond to a “night procedure”. FIG. 2A1 shows a time line which may correspond to a day procedure according to some embodiments of the invention. For example, a subject may begin a low residue diet two days before the examination. On the day before the examination, the subject may have his last meal during the morning or at noon; a laxative may be taken, for example, two hours and eight hours after the last meal. According to other embodiments, for example, a subject may ingest approximately three or four litters of Poly Ethylene Glycol (PEG), e.g., approximately eight hours after the last meal. In some embodiments, a stimulant may be taken approximately six or seven hours before an in-vivo imaging device is swallowed or otherwise inserted into the body. In some embodiments, most of the emptying procedure may be completed during the night before the examination, and the examination itself (e.g., obtaining images of the GI tract) may be performed during the day.

In some embodiments, approximately two to four hours after the insertion of the in-vivo imaging device into the GI tract, the procedure may be boosted by ingesting additional dose of gut lavage, for example, PEG (e.g., approximately one liter of PEG) and/or a laxative (e.g., approximately 30 milliliters of sodium phosphate). In some embodiments, the subject may eat again approximately six hours after insertion of the in-vivo imaging device. In some embodiments, the examination may be completed approximately eight to ten hours after ingestion of the in-vivo imaging device.

In some embodiments, a booster, for example, PEG, may be taken only after the in-vivo imaging device exits the stomach.

In the embodiment described in FIG. 2A2 a patient is put on a liquid diet on the day before the imaging device ingestion and is given a laxative (e.g., sodium phosphate) at the end of that day and at the beginning of the next morning. A prokinetic (e.g., Tegaserod™) may be administered later that morning after which a swallowable imaging device is ingested. The patient is given another dose of prokinetic approximately two hours after ingestion of the imaging device after which the patient may eat. A stimulant (e.g., Bisacodyl™) is given to the patient at the end of the day.

Colon cleansing procedures according to embodiments of the invention are designed to maintain colon cleansing level and to propel the imaging device (such as a capsule) efficiently. Procedures according to some embodiments may include:

Prokinetic agents taken approximately 1 hour prior to capsule ingestion and/or a few hours following ingestion; Additional laxative dose (boost) that is taken ˜2 h post capsule ingestion, typically after the capsule exits the stomach; use of a prokinetic agent; timing meal intake and stimulant intake (e.g. use of suppository stimulant).

In some embodiments, utilizing a day procedure, for example, as described with reference to FIG. 2A, may allow an advantage of having most of the fasting period during the night. Other advantages and benefits may be possible.

FIG. 2B shows a time line which may correspond to a night procedure according to some embodiments of the invention. A subject begins a low residue diet, for example, two days before the examination. On the day before the examination, the subject may have his last meal in the morning. Then, a laxative may be taken, for example, in two doses, e.g., at approximately 14:00 and 20:00. A stimulant or prokinetic agent may be ingested approximately one hour prior to insertion of the in-vivo imaging device. Then, the in-vivo device may be ingested or otherwise inserted into the GI tract. In accordance with some embodiments, the examination may be performed during the night. The subject may wake up at approximately 5 or 6 a.m. in the day after. Optionally, the procedure may be boosted by ingesting additional dose of gut lavage, for example, PEG (e.g., approximately one liter of PEG) and/or a laxative (e.g., approximately 30 milliliters of sodium phosphate), and the subject may then eat breakfast. The procedure may be completed approximately by noon time.

In some embodiments, a night procedure may provide the advantages of, for example, a shortened and easy fasting period (e.g., while the subject is asleep), and/or improved or optimal utilization of colon activity peaks. For example, a night procedure may utilize colon natural reflexes, e.g., to morning awakening and/or food intake. The night procedure may further allow the subject to go through most of the procedure in a friendly environment, e.g., at home. Another possible advantage of the night procedure may include, for example, the lying down position of the subject, which may allow a good or better view of otherwise difficult-to-view areas, for example, the ceacum. In some embodiments, the results of the examination may be obtained in the morning directly after the examination, thereby allowing a follow-up examination that same day, if necessary. Other benefits or advantages may be possible.

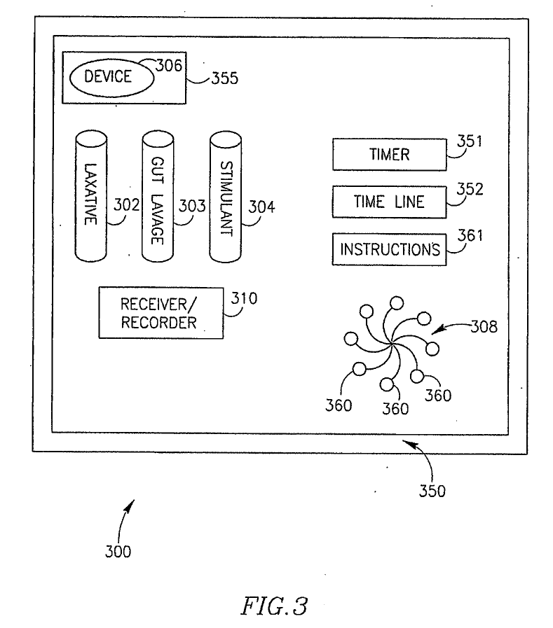

FIG. 3 schematically illustrates a kit 300 according to some embodiments of the invention. Kit 300 may include, for example, a laxative 302, a gut lavage 303, a stimulant or prokinetic agent 304, an autonomous in-vivo imaging device 306 (e.g., a swallowable in-vivo imaging capsule), a set of receiving antennas 308 (e.g., a portable or wearable set), and a receiver/recorder 310 able to receive and/or record images transmitted from the in-vivo imaging device. In some embodiments, kit 300 may optionally include a timer 351 and/or a timeline 352, e.g., for assisting the subject to timely go through the procedure. The components of kit 300 may be, for example, provided or enclosed within a packaging 350, e.g., a box, a container, a case, a bag, a carrying bag, a pouch, a purse, a suitcase or a mini-suitcase, or other enclosure.

In some embodiments, laxative 302 may include, for example, Soffodex™ e.g., available from Dexcel Ltd., Dexxon Ltd. and/or Dexcel Pharma Technologies Ltd., all of which from Israel, as described at <www.Dexxon.co.il>; for example, a solution having disodium hydrogen phosphate (e.g., approximately 0.9 grams or 5 milliliters) and sodium dihydrogen phosphate (e.g., approximately 2.4 grams or 5 milliliters). In some embodiment, laxative 302 may include, for example, Phospho-Soda® Oral Saline Laxative, available from Fleet®, as described at <www.PhosphoSoda.com>. Other suitable laxatives may be used.

In some embodiments, laxative 302 may include PEG-based products, for example: Meroken or Meroken New (available from Taro of Israel, <www.Taro.co.il>, e.g., bottled powder having 315.000 grams of Polyethylene Glycol, 4.2840 grams of Sodium Bicarbonate, 8.4240 grams of Sodium Chloride, and 1.1175 grams of Potassium Chloride); GoLYTELY® (available from BrainTree Laboratories Inc. of Braintree, Mass., <www.NyLYTELY.com>, e.g., a powder having 236 grams of Polyethylene Glycol 3350, 22.74 grams of Sodium Sulfate (anhydrous), 6.74 grams of Sodium Bicarbonate, 5.86 grams of Sodium Chloride, and 2.97 grams of Potassium Chloride); NuLYTELY® (available from BrainTree Laboratories Inc. of Braintree, Mass., <www.NyLYTELY.com>, e.g., a powder having 420 grams of Polyethylene Glycol 3350, 5.72 grams of Sodium Bicarbonate, 11.2 grams of Sodium Chloride, 1.48 grams of Potassium Chloride, and optionally a 2.0 grams of flavor substance); or other suitable laxatives or a combination thereof.

Kit may 300 may include one or more portions of the laxative, lavage or other solutions. A portion may include, for example, approximately 30 to 45 milliliter of sodium phosphate, laxative or approximately one liter of PEG, or other suitable quantities and/or substances. Multiple portions may be stored or mixed in a single bottle or receptacle, or may be stored in separate bottles or other receptacles.

In some embodiments, stimulant 304 may include, for example, Bisacodyl, Senna, Castor Oil, or other suitable substances. Kit 300 may include one or more portions of the stimulant 304.

In some embodiments, the autonomous in-vivo imaging device 306, which may be, for example, swallowable, may be provided in a blister or other appropriate packaging 355. In some embodiments, packaging 355 may include a magnet, which may deactivate the device 306 when in proximity to the device 306; for example, removal of device 306 from packaging 355 may cause device 306 to be activated and/or operational. Other suitable activation methods may be used. In some embodiments, device 306 may be swallowed by a subject, for example, together with a cup of water. Other suitable ways of inserting the device 306 may be used.

In some embodiments, prior to swallowing the device 306, the subject may wear the set of receiving antennas 308 having one or more antennas 360, e.g., eight antennas. The antennas 360 may be implemented as part of a jacket, belt, or other garment or article of clothing which, when placed over the subject's anatomy, may position the antennas 360 for receiving image data transmitted from the device 306 from within the subject's body. In some embodiments, the antennas 360 may be provided with adhesive means, for example, a glue, a bonding material, or the like, e.g., for attaching the antennas 360 directly to the subject's body. In some embodiments, an instructions leaflet 361 may be provided, e.g., to ensure correct positioning of the antennas 360 on the subject's body. The instructions article 361 may be implemented, for example, using an instructions sheet, a manual, a booklet, a map, a chart, a flow-chart, a table, a checklist, a drawings, a textual explanation, a graphical explanation, an audio explanation, a video explanation, an article having stored thereon instructions (e.g., memory unit, audio cassette, video cassette, Compact Disk, CD-ROM, DVD, or the like), or the like.

In some embodiments, receiver/recorder 310 may be attached to the set of receiving antennas 308. Receiver/recorder 310 may, for example, receive image data through the antennas 308 and may record data onto a hard disc or other suitable memory device, e.g., a removable memory unit, a Flash memory, or the like. Image data stored in receiver/recorder 310 may be transferred to a data processor, for example, in a professional's workstation. For example, receiver/recorder 310 may be taken off the subject's body after the examination, and may be taken to a physician who may connect the receiver/recorder to a personal computer or workstation (e.g., having a data processor, a storage unit, and a display unit) via a suitable data link, e.g., a serial interface, a parallel interface, a USB interface, or other wired or wireless interfaces. In some embodiments, receiver/recorder 310 may, for example, include processing capabilities and/or a display (e.g., LCD display) from displaying image data.

In some embodiments, kit 300 may include color coding of components, e.g., for user-friendly operation. For example, laxative(s) 302 or other substances intended for taking prior to swallowing of the device 306 may be marked using a first color, and laxative(s) 302 or other substances intended for taking after the device 306 is swallowed may be marked using a second, different, color. Other suitable color schemes may used, for example, to facilitate the use of components of kit 300 by a subject.

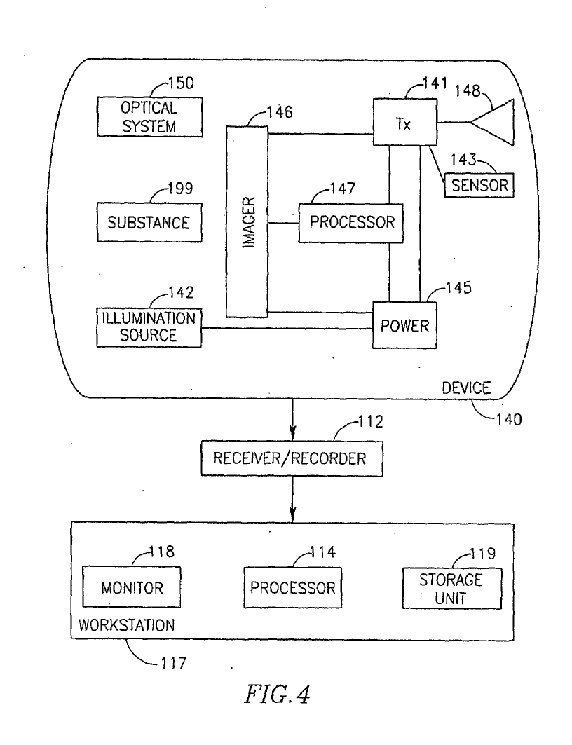

FIG. 4 shows a schematic illustration of an in-vivo system in accordance with some embodiments of the present invention. One or more components of the system may be used in conjunction with, may be operatively associated with, the devices and/or components described above, or other in-vivo devices in accordance with embodiments of the invention.

In some embodiments, the system may include a device 140 having a sensor, e.g., an imager 146, one or more illumination sources 142, a power source 145, and a transmitter 141. In some embodiments, device 140 may be implemented using a swallowable capsule, but other sorts of devices or suitable implementations may be used. Outside a patient's body may be, for example, an external receiver/recorder 112 (including, or operatively associated with, for example, an antenna or an antenna array), a storage unit 119, a processor 114, and a monitor 118. In some embodiments, for example, processor 114, storage unit 119 and/or monitor 118 may be implemented as a workstation 117, e.g., a computer or a computing platform.

Transmitter 141 may operate using radio waves; but in some embodiments, such as those where device 140 is or is included within an endoscope, transmitter 141 may transmit/receive data via, for example, wire, optical fiber and/or other suitable methods. Other known wireless methods of transmission may be used. Transmitter 141 may include, for example, a transmitter module or sub-unit and a receiver module or sub-unit, or an integrated transceiver or transmitter-receiver.

Device 140 typically may be or may include an autonomous swallowable capsule, but device 140 may have other shapes and need not be swallowable or autonomous. Embodiments of device 140 are typically autonomous, and are typically self-contained. For example, device 140 may be a capsule or other unit where all the components are substantially contained within a container or shell, and where device 140 does not require any wires or cables to, for example, receive power or transmit information. In some embodiments, device 140 may be autonomous and non-remote-controllable; in another embodiment, device 140 may be partially or entirely remote-controllable.

In some embodiments, device 140 may communicate with an external receiving and display system (e.g., workstation 117 or monitor 118) to provide display of data, control, or other functions. For example, power may be provided to device 140 using an internal battery, an internal power source, or a wireless system able to receive power. Other embodiments may have other configurations and capabilities. For example, components may be distributed over multiple sites or units, and control information or other information may be received from an external source.

In some embodiments, device 140 may include an in-vivo video camera, for example, imager 146, which may capture and transmit images of, for example, the GI tract while device 140 passes through the GI lumen. Other lumens and/or body cavities may be imaged and/or sensed by device 140. In some embodiments, imager 146 may include, for example, a Charge Coupled Device (CCD) camera or imager, a Complementary Metal Oxide Semiconductor (CMOS) camera or imager, a digital camera, a stills camera, a video camera, or other suitable imagers, cameras, or image acquisition components.

In some embodiments, imager 146 in device 140 may be operationally connected to transmitter 141. Transmitter 141 may transmit images to, for example, external transceiver or receiver/recorder 112 (e.g., through one or more antennas), which may send the data to processor 114 and/or to storage unit 119. Transmitter 141 may also include control capability, although control capability may be included in a separate component, e.g., processor 147. Transmitter 141 may include any suitable transmitter able to transmit image data, other sensed data, and/or other data (e.g., control data) to a receiving device. Transmitter 141 may also be capable of receiving signals/commands, for example from an external transceiver. For example, in some embodiments, transmitter 141 may include an ultra low power Radio Frequency (RF) high bandwidth transmitter, possibly provided in Chip Scale Package (CSP).

In some embodiment, transmitter 141 may transmit/receive via antenna 148. Transmitter 141 and/or another unit in device 140, e.g., a controller or processor 147, may include control capability, for example, one or more control modules, processing module, circuitry and/or functionality for controlling device 140, for controlling the operational mode or settings of device 140, and/or for performing control operations or processing operations within device 140. According to some embodiments, transmitter 141 may include a receiver which may receive signals (e.g., from outside the patient's body), for example, through antenna 148 or through a different antenna or receiving element. According to some embodiments, signals or data may be received by a separate receiving device in device 140.

Power source 145 may include one or more batteries or power cells. For example, power source 145 may include silver oxide batteries, lithium batteries, other suitable electrochemical cells having a high energy density, or the like. Other suitable power sources may be used. For example, power source 145 may receive power or energy from an external power source (e.g., an electromagnetic field generator), which may be used to transmit power or energy to in-vivo device 140.

In some embodiments, power source 145 may be internal to device 140, and/or may not require coupling to an external power source, e.g., to receive power. Power source 145 may provide power to one or more components of device 140 continuously, substantially continuously, or in a non-discrete manner or timing, or in a periodic manner, an intermittent manner, or an otherwise non-continuous manner. In some embodiments, power source 145 may provide power to one or more components of device 140, for example, not necessarily upon-demand, or not necessarily upon a triggering event or an external activation or external excitement.

Optionally, in some embodiments, transmitter 141 may include a processing unit or processor or controller, for example, to process signals and/or data generated by imager 146. In another embodiment, the processing unit may be implemented using a separate component within device 140, e.g., controller or processor 147, or may be implemented as an integral part of imager 146, transmitter 141, or another component, or may not be needed. The processing unit may include, for example, a Central Processing Unit (CPU), a Digital Signal Processor (DSP), a microprocessor, a controller, a chip, a microchip, a controller, circuitry, an Integrated Circuit (IC), an Application-Specific Integrated Circuit (ASIC), or any other suitable multi-purpose or specific processor, controller, circuitry or circuit. In some embodiments, for example, the processing unit or controller may be embedded in or integrated with transmitter 141, and may be implemented, for example, using an ASIC.

In some embodiments, imager 146 may acquire in-vivo images continuously, substantially continuously, or in a non-discrete manner, for example, not necessarily upon-demand, or not necessarily upon a triggering event or an external activation or external excitement; or in a periodic manner, an intermittent manner, or an otherwise non-continuous manner.

In some embodiments, transmitter 141 may transmit image data continuously, or substantially continuously, for example, not necessarily upon-demand, or not necessarily upon a triggering event or an external activation or external excitement; or in a periodic manner, an intermittent manner, or an otherwise non-continuous manner.

In some embodiments, device 140 may include one or more illumination sources 142, for example one or more Light Emitting Diodes (LEDs), “white LED”, or other suitable light sources. Illumination sources 142 may, for example, illuminate a body lumen or cavity being imaged and/or sensed. An optional optical system 150, including, for example, one or more optical elements, such as one or more lenses or composite lens assemblies, one or more suitable optical filters, or any other suitable optical elements, may optionally be included in device 140 and may aid in focusing reflected light onto imager 146, focusing illuminated light, and/or performing other light processing operations.

In some embodiments, illumination source(s) 142 may illuminate continuously, or substantially continuously, for example, not necessarily upon-demand, or not necessarily upon a triggering event or an external activation or external excitement. In some embodiments, for example, illumination source(s) 142 may illuminate a pre-defined number of times per second (e.g., two or four times), substantially continuously, e.g., for a time period of two hours, four hours, eight hours, or the like; or in a periodic manner, an intermittent manner, or an otherwise non-continuous manner.

In some embodiments, the components of device 140 may be enclosed within a housing or shell, e.g., capsule-shaped, oval, or having other suitable shapes. In some embodiments, the housing or shell may be substantially transparent or semi-transparent, and/or may include one or more portions, windows or domes which may be substantially transparent or semi-transparent. In some embodiments, for example, one or more illumination source(s) 142 within device 140 may illuminate a body lumen through a transparent or semi-transparent portion, window or dome; and light reflected from the body lumen may enter the device 140, for example, through the same transparent or semi-transparent portion, window or dome, or, optionally, through another transparent or semi-transparent portion, window or dome, and may be received by optical system 150 and/or imager 146. In some embodiments, for example, optical system 150 and/or imager 146 may receive light, reflected from a body lumen, through the same window or dome through which illumination source(s) 142 illuminate the body lumen.

Data processor 114 may analyze the data received via external receiver/recorder 112 from device 140, and may be in communication with storage unit 119, e.g., transferring frame data to and from storage unit 119. Data processor 114 may provide the analyzed data to monitor 118, where a user (e.g., a physician) may view or otherwise use the data. In some embodiments, data processor 114 may be configured for real time processing and/or for post processing to be performed and/or viewed at a later time. In the case that control capability (e.g., delay, timing, etc) is external to device 140, a suitable external device (such as, for example, data processor 114 or external receiver/recorder 112 having a transmitter or transceiver) may transmit one or more control signals to device 140. Monitor 118 may include, for example, one or more screens, monitors, or suitable display units. Monitor 118, for example, may display one or more images or a stream of images captured and/or transmitted by device 140, e.g., images of the GI tract or of other imaged body lumen or cavity. Additionally or alternatively, monitor 118 may display, for example, control data, location or position data (e.g., data describing or indicating the location or the relative location of device 140), orientation data, and various other suitable data. In some embodiments, for example, both an image and its position (e.g., relative to the body lumen being imaged) or location may be presented using monitor 118 and/or may be stored using storage unit 119. Other systems and methods of storing and/or displaying collected image data and/or other data may be used.

Typically, device 140 may transmit image information in discrete portions. Each portion may typically correspond to an image or a frame; other suitable transmission methods may be used. For example, in some embodiments, device 140 may capture and/or acquire an image once every half second, and may transmit the image data to external receiver/recorder 112. Other constant and/or variable capture rates and/or transmission rates may be used.

Typically, the image data recorded and transmitted may include digital color image data; in alternate embodiments, other image formats (e.g., black and white image data) may be used. In some embodiments, each frame of image data may include 256 rows, each row may include 256 pixels, and each pixel may include data for color and brightness according to known methods. For example, a Bayer color filter may be applied. Other suitable data formats may be used, and other suitable numbers or types of rows, columns, arrays, pixels, sub-pixels, boxes, super-pixels and/or colors may be used.

Optionally, device 140 may include one or more sensors 143, instead of or in addition to a sensor such as imager 146. Sensor 143 may, for example, sense, detect, determine and/or measure one or more values of properties or characteristics of the surrounding of device 140. For example, sensor 143 may include a pH sensor, a temperature sensor, an electrical conductivity sensor, a pressure sensor, or any other known suitable in-vivo sensor.

In some embodiments, one or more substances, solutions, laxatives, stimulants, or other materials 199 may optionally be stored inside device 140, and may be released within a body lumen (e.g., the GI tract or the colon) or a pre-defined area thereof when the device 140 reaches that body lumen or area.

According to one embodiment of the invention the in vivo imaging device has a any volume to weight ratio that enables it essentially to float.

According to one embodiment of the invention the in vivo imaging device has a specific gravity (SG) of about 0.7-0.98 in the colon's fluid. In an embodiment of the invention the body of the device may be filled with a substance lighter than the body lumen liquid, such as gaseous CO₂, O₂ or air.

In an embodiment of the invention, there is provided a method of adjusting the weight/volume ratio of a capsule to the specific gravity of the liquid in the lumen.

In an embodiment of the invention the lumen liquid may be enriched with high molecular substances.

In another embodiment of the invention the lumen liquid may be enriched with low molecular substances.

In another embodiment of the invention, the in vivo imaging device has a and the buoyant body is an inflatable buoy such as described in United States Publication No. US-2003-001828. The buoy may be packaged such that it is not buoyant while in packaging. However, release of the buoy from its packaging lends buoyancy to the in vivo imaging device. The buoy may be released from its packaging at a desired location or point in time, such that the in vivo imaging device may acquire buoyancy according to specific requirements. For example, the floatable in vivo imaging device according to an embodiment of the invention may be ingested and moved by peristalsis through the small intestine while its buoy is packaged. When the device enters the large intestine the buoy is released from its packaging and the device can then float in the bulk of liquid in the large intestine and be carried by the bulk of liquid to all areas of the large intestine. Thus, the device is moved through the large intestine, effectively imaging the lumen.

In one embodiment the patient's large intestine is initially cleared of its contents, for example by inducing bowel movement by administering a laxative or an enema. Further, the patient's large intestine is filled with a liquid, for example by drinking high osmolarity liquids, which retain liquids within the large intestine for longer periods. Typically, a liquid loaded intestine and/or additional laxatives, administered while the in vivo imaging device is in the large intestine, will induce bowel movement and cause a flow of the bulk of liquids in the large intestine. The induced flow will enhance movement of the sensor system through the large intestine, thereby facilitating sensing of most areas of the lumen.

According to one embodiment of the invention an imaging device for imaging the GI tract can be moved through the large intestine by utilizing, for example, a high osmotic pressure composition that is essentially not absorbed by the intestine. According to other embodiments an object other than an imaging device, for example a device for sustained release of medicaments to the colon, can be moved through the large intestine by utilizing a high osmotic pressure composition that is essentially not absorbed by the intestine. Objects moved through the large intestine utilizing a high osmotic pressure composition according to an embodiment of the invention may or may not be floatable.

The device is ingested by a patient and traverses the small intestine pushed along by natural peristalsis. When the device 10 reaches the cecum it typically remains in the cecum for long periods of time.

A high osmotic pressure composition that is essentially not absorbed from the intestine is administered to the patient and progresses through the small intestine arriving at the cecum. For example the osmotic pressure of Gastrografin at 37° C. is 55.1 atm and its osmolality is 2.15 (osm/kg H₂O).

If left to the natural action of the colon muscles, the device 10 would move slowly and erratically through the colon depleting the device power while possibly being unable to obtain a sufficient number of images for efficiently monitoring and diagnosing the colon. Thus, the invention enables wireless imaging of the colon which was formerly not easily achieved.

Various aspects of the various embodiments disclosed herein are combinable with the other embodiments disclosed herein.

Although portions of the discussion herein may relate to an imager or an image sensor, embodiments of the invention are not limited in this regard; such imager or image sensor may include, for example, a detector, a sensor, a photodiode, a florescence device, an electrochemical sensing device, a magnetic field sensing device, a spectrophotometer, an image sensor, a Charge Coupled Device (CCD) camera or imager, a Complementary Metal Oxide Semiconductor (CMOS) camera or imager, a digital camera, a stills camera, a video camera, a light sensor; a device capable of detecting or sensing one or more colors, intensities, hues, brightness, contrast, and/or other parameters or characteristic; a device sensitive to one or more colors or able to detect one or more colors; a device capable of detecting one or more color changes; a device sensitive to color changes; or the like

A device, system and method in accordance with some embodiments of the invention may be used, for example, in conjunction with a device which may be inserted into a human body. However, the scope of the present invention is not limited in this regard. For example, some embodiments of the invention may be used in conjunction with a device which may be inserted into a non-human body or an animal body.

It is noted that while some exemplary embodiments are explained in detail herein, the invention is not limited in this regard, and other embodiments and/or implementations of a broad field-of-view imaging device are also within the scope of the invention. While certain features of the invention have been illustrated and described herein, many modifications, substitutions, changes, and equivalents may occur to those of ordinary skill in the art The claims are therefore intended to cover all such modifications and changes as fall within the true spirit of the invention.

1. A method for an in vivo examination, the method comprising:
substantially emptying a subject's colon from content;
administering to the subject a prokinetic agent;
inserting a wireless imaging device into the subject's GI tract; and
receiving images transmitted from said imaging device.

2. The method according to claim 1 comprising recording the received image data.

3. The method according to claim 2 wherein recording is onto a removable memory device.

4. The method according to claim 1 comprising displaying images received from the imaging device.

5. The method according to claim 1 wherein emptying a subject's colon comprises ingesting laxatives and stimulants.

6. The method according to claim 1 wherein inserting a wireless imaging device is by swallowing the device.

7. The method according to claim 1 comprising obtaining images of a subject's colon on an autonomous in vivo imaging device.

8. The method according to claim 1 wherein receiving images is by an external receiving unit.

9. A kit for in vivo examination, the kit comprising: a laxative; a wireless imaging capsule; a receiving antenna and a recorder.

10. The kit according to claim 9 wherein the laxative includes PEG and/or sodium phosphate.

11. The kit according to claim 9 comprising a plurality of portions of laxatives.

12. The kit according to claim 9 comprising a stimulant.

13. The kit according to claim 9 wherein the wireless capsule comprises a housing which encloses an illumination source, an optical system, an imager and a transmitter.

14. The kit according to claim 9 comprising 1-8 receiving antennas.

15. The in-vivo examination kit of claim 12, wherein at least one of the laxative and the stimulant is color-coded.

16. The in-vivo examination kit of claim 9, further comprising:
an instruction sheet having instructions to ingest prokenitic, to wait a pre-defined time period and to insert an imaging capsule.

17. The in-vivo examination kit of claim 9, further comprising:
an instructions leaflet having instructions to insert the autonomous in-vivo imaging device at night time and at least thirty minutes after administering the stimulant.

18. The in-vivo examination kit of claim 9, wherein the instructions article comprises an instruction to ingest a meal and a booster laxative at least five hours after inserting the autonomous in-vivo imaging device.

19. The in-vivo examination kit of claim 9, further comprising:
a packaging to enclose the a wireless imaging capsule; a receiving antenna and a recorder and the laxative.

20. The in-vivo examination kit of claim 17, wherein the wireless imaging capsule comprises an imager, an illumination source, and a wireless transmitter all encapsulated in a single housing.
